(19) United States
(12) Patent Application Publication
(10) Pub. No.: US 2020/0214902 A1
(43) Pub. Date: Jul. 9, 2020

(54) SYSTEMS AND METHODS FOR MONITORING A WEARABLE DEVICE

(71) Applicant: VIECONNECT SAS, Toulouse (FR)

(72) Inventors: Guillermo ROMERO, Phoenix, AZ (US); Dimitri TINTIKAKIS, Toulouse (FR); Aristidis TINTIKAKIS, Toulouse (FR)

(73) Assignee: VIECONNECT SAS, Toulouse (FR)

(21) Appl. No.: 16/627,974

(22) PCT Filed: Jul. 10, 2018
(86) PCT No.: PCT/US18/41372

§ 371 Date: Dec. 31, 2019

Related U.S. Application Data
(60) Provisional application No. 62/532,490, filed on Jul. 14, 2017.

(51) Int. Cl. A61F 13/42

(52) U.S. Cl. CPC A61F 13/42; A61F 2013/424

(57) ABSTRACT

A wetness or saturation and temperature, and motion, posture or orientation detecting system in an undergarment. A wireless module can comprise a housing, a cover, and a controller. The controller can comprise environmental sensors, a motion analyzer, and a memory medium. The environmental sensors can be a temperature sensor, a humidity sensor, a pressure sensor that can monitor and detect environmental conditions surrounding it within the undergarment or other article of clothing. The wireless module can further comprise a microcontroller unit (“MCU”) and a communications module. The cover can have a thru hole substantially positioned over the environmental sensor wherein the thru hole allows the environmental sensor access to the outside elements. The wireless module can further comprise a filter element, and a locking ring wherein the filter element can be affixed to the cover. The cover can be attached to the housing encapsulating the controller, battery, and bushing.

### CROSS REFERENCE TO RELATED APPLICATIONS

The present application claims the benefit under 35 U.S.C. 119 of U.S. Provisional Patent Application Ser. No. 62/532,490 filed Jul. 14, 2017. The U.S. Provisional Patent Application Ser. No. 62/532,490 is hereby incorporated by reference in its entirety.

### BACKGROUND OF THE INVENTION

### Field of the Invention

The present invention relates to a humidity sensing device and more particularly to a remote monitoring system for indicating the need for an article of clothing to be changed when saturated or soiled.

### Description of Related Art

In nursing homes, mental institutions, care centers, or other care giving facilities an individuals' incontinent undergarment is usually checked on every few hours. If the individual releases their urine or other bodily fluid right after being checked this condition can go unnoticed for several hours or more. In addition, a significant percentage of people are not able to effectively communicate episodes of incontinence or loss of bladder control with their caregivers. As a result, many people experience long periods of time in which their clothing or incontinent undergarment is saturated in urine which exposes that individual to harsh and irritating conditions caused by urine and other bodily fluids. A condition commonly known as “diaper rash” or other infections may develop making it even more critical for the caregiver to change the patient's clothing or incontinence undergarment. Moreover, individuals that live in nursing homes are usually woken up every few hours, interrupting their sleep, for the caregiver to check their incontinent undergarment.

Various types of devices for monitoring, detecting and notifying of the condition of a diaper, bedding, or adult incontinence undergarment exist in the art. The general principle of a wetness detection system that is known in the art usually activates an audible or visible alarm when two electrodes complete a circuit when in the presence of urine or other bodily fluids. This usually is accomplished by detecting some sort of threshold of wetness within an incontinence undergarment. Several attempts have been made in the past to address moist incontinence undergarments. For example, Kline et al., U.S. Pat. No. 5,264,830 discloses a sensing device which uses an alarm positioned on the outside of the diaper. The device includes a small battery-powered audible alarm connected to an electrical circuit having contacts that when wet completes a circuit and sounds an alarm. The problem with this device is that the caregiver is not usually in the same room as the individual to hear an audible alarm or see a visual indicator. In addition, the device usually requires the user to wear waterproof pants that adds to the wearer's clothing making he/she uncomfortable when wearing the undergarment.

While these and similar devices have been somewhat successful in signaling an event within an undergarment, they still tend to exhibit numerous problems and shortcoming inherent with the respective designs. Accordingly, a significant advance in the art could be realized if a device could be developed that could monitor, detect, and then alert a caretaker remotely when the individual has soiled their undergarment. The device may be small and compact as to not interfere with or cause any discomfort to the individual, yet should not be readily swallowed or removed from the undergarment. In addition, the device may be sensitive enough to detect the slightest humidity, temperature or orientation change, and then promptly alert the caretaker after detecting saturation, elevated temperature, motion or a posture change event.

Accordingly, there exists a continuing and unaddressed need for a device that can monitor, detect and notify a caretaker or family member when there is an event with an individual's undergarment. In addition, advancements in the art allow the caretaker, or family member to track remotely the initial wetness event from when the undergarment is changed through an app or webpage on a smartphone, or computer web portal.

### SUMMARY OF THE INVENTION

Aspects disclosed herein relates to a system and methods for monitoring, detecting, and notifying an individual when someone has a saturation event in their undergarment or temperature, posture or orientation change of his/her body. A wetness, motion and orientation detecting system can comprise a wireless module with a controller accommodated within a housing at least partially separating the controller from the environment to keep the controller and any power source for the controller from getting wet and shorting. An environmental sensor at least partially in contact with the environment around the wireless module detects at least one feature of the environment such as humidity, temperature, or the presence or absence of a particular compound or molecule. A motion sensor, such as an accelerometer, or compass detects a motion or an orientation of the module and may relate that motion or orientation to the motion or orientation of the wearer of the wireless module. A transceiver may be included to allow the module to transmit information to and receive information or direction from a remote device, such as a wireless communications computer, a cell phone or other mobile communications device. A memory medium is used to provide executable instruction to the controller and/or to store data collected by the environmental or motion and orientation sensors.

In a particular embodiment, the environmental sensor is within the housing with the controller and the housing has at least one thru hole substantially positioned over the environmental sensors, with a bushing substantially positioned between the environmental sensors and the housing. A filter element and a locking ring may also be provided, wherein the filter element is affixed into the bushing and the locking ring secures the filter element substantially between the housing and the locking ring.

The housing may be sealed by a cover encapsulating the controller and a power supply to ensure that the wireless module is resistant to fluids. The power supply may be a rechargeable or replaceable battery. The controller may be configured to intermittently activate the battery to conserve energy and extend battery life.

The wireless module may be secured into a secondary pouch wherein the secondary pouch can be removably attached to an undergarment. The secondary pouch may have a plurality of holes on its surface to allow fluids or humidity to penetrate the secondary pouch. The wireless module according to claim 2, wherein the remote device is a wireless communications computer or a cell phone or other mobile communications device.

The wireless module may be used to determine whether there is an environmental change with the environmental sensors, and then transmit that state to a remote central hub, local or cloud server or other device.

The objects of the invention are further accomplished by a method of detecting wetness or saturation of an undergarment, and motion, posture or orientation of the wearer through securing a wireless module to a secondary pouch or directly attaching it to an interior of an undergarment, monitoring the environmental conditions of the undergarment and the motion, posture or orientation of the wearer and transmitting data related to the environmental characteristics and the wearer's motion, posture or orientation from the wireless module to remote device where the data are stored. Alerts can be created based on the data alerting a caregiver when the undergarment becomes wet, saturated or soiled or the wearer of the undergarment is moving, has changed posture or fallen down.

Historical data such as the last known environmental condition or motion, posture or orientation characteristic may be stored and changes to that environmental condition or motion, posture or orientation will create an alert that is conveyed to a caregiver. For example, an alert may be created when the undergarment changes from dry to wet or saturated or an abnormal motion or a posture change or a fall of the wearer has been detected. The historical data may also be used to keep track of the wearer of the undergarment and frequency of abnormal activity, falls and/or soiling of the undergarment. A display of the current status of the wearer may be accessed by a caregiver at any time.

The alerts or information may be transmitted to the caregiver or other personnel via a mobile communications device such as a smartphone, personal digital assistance, a tablet device, a table PC, a laptop, a smart book, or an ultra-book. Software or other data stored on the wireless module may be updated remotely through a remote central hub, a local or cloud server, or other remote device.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 illustrates an isometric view of a wetness or saturation and motion, posture and orientation detecting system within an undergarment in accordance to one, or more embodiments;

FIG. 2 illustrates an exploded isometric view of a wetness or saturation and motion, posture and orientation detecting system within an undergarment in accordance to one, or more embodiments;

FIG. 2B illustrates an exploded isometric view of another embodiment of a wetness or saturation and motion, posture and orientation detecting system within an undergarment in accordance to one, or more embodiments;

FIG. 3 illustrates a cross-sectional view of a wetness or saturation and motion, posture and orientation detecting system within an undergarment in accordance to one, or more embodiments;

FIG. 4A illustrates a process flow diagram for depicting a method or system for detecting humidity or saturation by inserting a wireless module into an undergarment in accordance to one, or more embodiments;

FIG. 4B illustrates a process flow diagram for depicting a method or system for detecting humidity or saturation by taping a wireless module into an undergarment in accordance to one, or more embodiments;

FIG. 5 illustrates a process flow diagram 500 depicting a method for detecting the state of the wireless module 100, in accordance with one, or more embodiments; and

FIG. 6 illustrates a process flow diagram 600 for depicting a method or system for monitoring, detecting and alerting when humidity or saturation is detected in an undergarment in accordance to one, or more embodiments.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

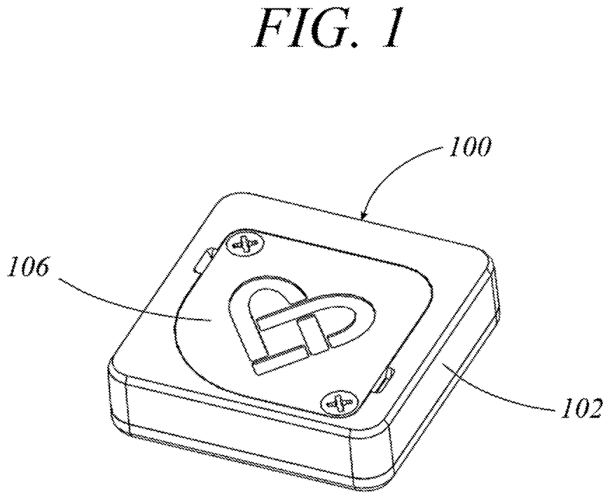

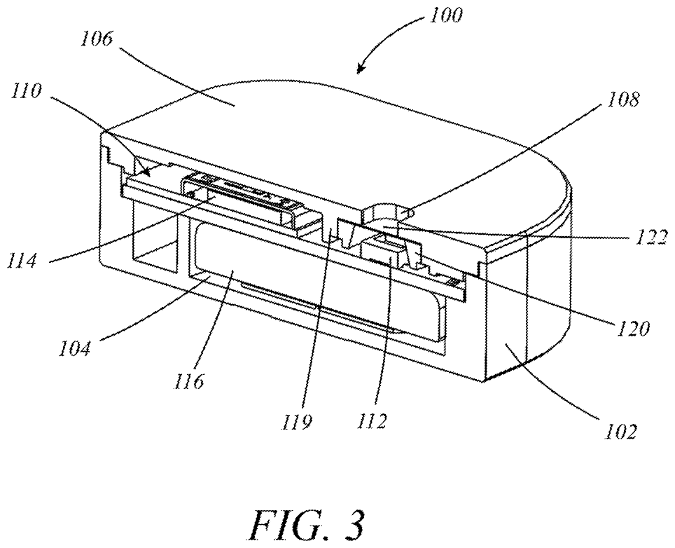

Referring generally to FIG. 1 through 3, which illustrates a wetness or saturation and motion, posture or orientation detecting system in an undergarment, shown generally at 100. In a preferred embodiment, a wireless module 100 can comprise a housing 102, a cover 106, and a controller 110. The controller 110 can comprise environmental sensors 112, a motion analyzer 116, and a memory medium. The environmental sensors can be any combination of a temperature sensor, a humidity sensor, a pressure sensor, a gas sensor, or the like that can monitor and detect environmental conditions proximal to the wireless module 100 within the undergarment or other article of clothing. The environmental sensor 112 can be a dual sensor module comprising both a humidity and temperature sensor, or can be a single separate sensor comprising of a humidity sensor, and a temperature sensor. In another embodiment, the environmental sensor can detect the presence of a given compound, such as, for example, urea, or another biological compound. The humidity sensor can be, for example, a relative (RH) humidity sensor, absolute humidity (moisture) sensor, or the like. The temperature sensor can be, for example, thermocouple, thermistor, resistance temperature detector, semiconductor based sensor, or the like. The motion analyzer 116 can be, but is not limited to a single axis, 2-axis, or a 3-axis digital accelerometer, optical, piezoelectric, capacitive, electromechanical, gyroscope, compass, or the like. The controller 110 base material can be manufactured out of a single sided, double sided, multilayered, flex, or the like printed circuit board (“PCB”).

In some embodiments, the wireless module 100 can further comprise a microcontroller unit (“MCU”) and a communications module which can be, for example, a BLUETOOTH Low Energy (BLE), BLUETOOTH module, Wi-Fi module, IEEE 802.15.4, Z-Wave, Single/Dual Mode Radio Chip or the like. The memory medium can be, for example, random access, flash, read only, or the like, and can store embedded software that provides commands to the MCU, communication module, environmental sensors, or motion analyzer, and stores and controls the data to be sent to a central hub. The MCU can execute program instructions that can be stored on the memory medium or on the MCU itself, such as, but not limited to, instructions on monitoring, detecting and transmitting when the undergarment has undergone a moisture event, and/or instructions on when the orientation of the wearer has changed wherein the orientation can be a fall, and/or battery notification, and/or when the wireless module is removed from and placed into an undergarment, or the like. In some embodiments, the module 100 can be configured to be paired with the intended user of the module 100. A user profile can be created that will be associated with the module 100. The user profile can include the user's age, sex, address or room number, medical condition, or any other information known by those skilled in the art.

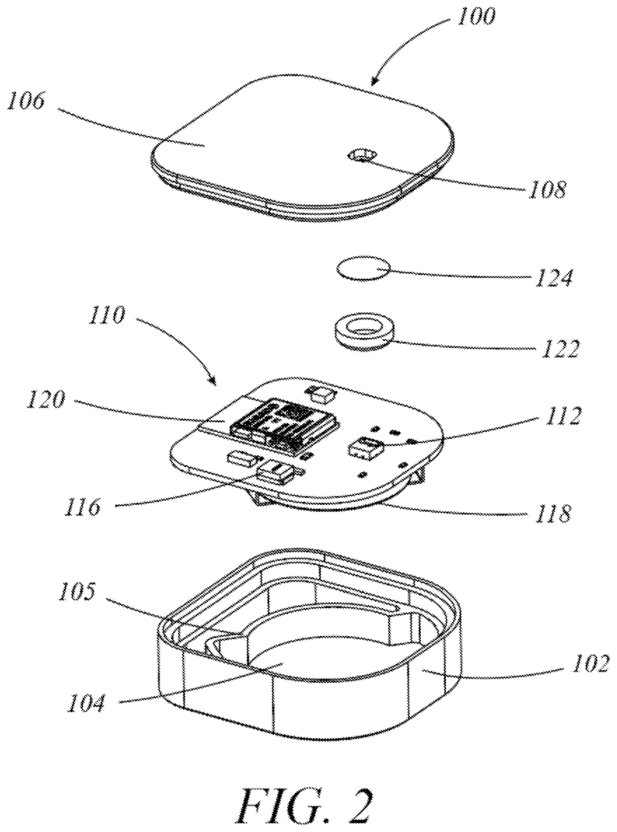

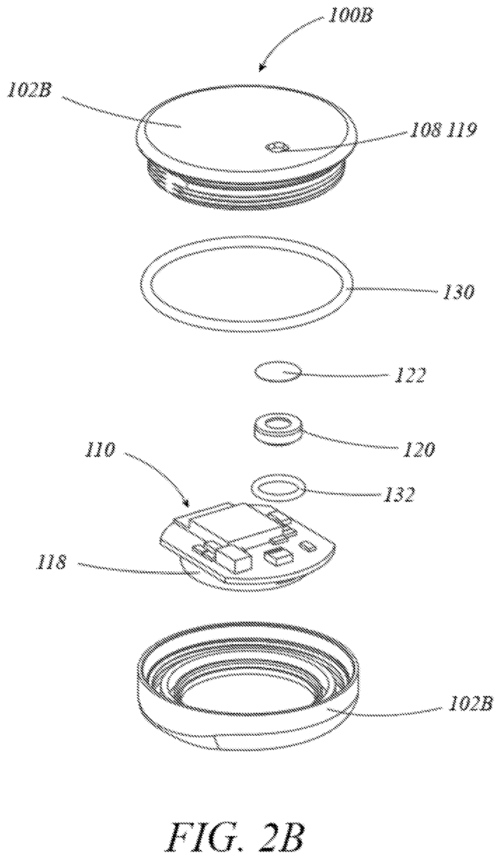

The controller 110 can further comprise a battery 118 which can be capable of powering a solid-state device. The battery 118 may include, for example, a lithium ion battery, a nickel cadmium battery, an alkaline battery, or the like. In certain embodiments, the battery 118 can be rechargeable by induction circuitry, allowing the user to remotely electrically charge via external induction circuitry. In another embodiment, the battery 118 is rechargeable wherein the wireless module can have a recharging port, allowing the user to electrically charge the battery. In certain embodiments, the housing 102 can include, but is not required to include, a recharging port that can be sealed from the outside environment, allowing the user to selectively access the recharging port to electrically charge the battery 118. In some embodiments, the controller 110 and the battery 118 can be placed into the housing 102 wherein the housing can have one or more slots comprising a battery slot 104, and a controller slot 105 to hold the battery and controller securely within the housing. The housing 102 can be circular, square, triangular, rectangular, or the like in shape having a thickness that can hold the controller 110 and the battery 118. In another embodiment, the housing 102B can be threaded around the inside circumference, and the cover 106B can be threaded around its outside circumference to allow the cover 106B to screw onto and encapsulate a cap O-ring 132 between the housing and the cover as shown in FIG. 2B creating a watertight seal.

In some embodiments, the cover 106 can have a thru hole 108 substantially positioned over the environmental sensor 112 wherein the thru hole allows the environmental sensor access to the outside elements, such as, humidity, temperature, liquids, pressure, gas or the like. The cover 106 can further comprise a bushing 119 located on the bottom side of the cover positioned substantially over the environmental sensor 112. The cover 106 can be circular, square, triangular, or the like in shape. The cover 106 and the housing 102 can be made from, but not limited to, plastics, metals, or other like materials. The wireless module 100 can further comprise a filter element 122, and a locking ring 120 wherein the filter element can be affixed to the cover 106 and can be substantially placed between the cover and the locking ring covering the thru hole. The bushing 119 can be substantially match the shape of the locking ring 120 wherein the locking ring can be pushed into the bushing by, for example, interference fit, press fit, loose fit, snap rings, or the like.

In particular embodiments, affixing a filter element 122 to a cover 106 can be defined as press fitting the locking ring 120 into a bushing 119 located on the cover 106 and substantially over the temperature and humidity sensor 112 with the filter element substantially between the locking ring and cover, clamping the filter element to the cover, gluing the filter element to the cover, or the like. The filter element 122 can allow vapor from fluids to pass through to the environmental sensor 112 on the controller 110 without allowing fluids to enter onto and get the controller wet. The filter element 122 can consist of hydrophobic aerogels, polytetrafluoroethylene (“PTFE”), silicone, or the like. The filter element 122 can be circular, square, triangular, rectangular, or the like in shape to completely fill the thru hole 108.

In some embodiments, the cover 106 can be attached to the housing 102 encapsulating the controller 110, battery 118, and bushing 119 wherein the cover can ultrasonic welded, glued, screwed onto (e.g., as shown in FIG. 2B), rubber sealed, O-rings placed into, gasket sealed or otherwise attached by methods that seal and keep the elements from penetrating and passing through the seal and onto the controller. In another embodiment, a filter O-ring 132 can be placed between the locking ring 120, bushing 108, and the controller 110 creating a barrier between the cover's 102B thru hole 108 as shown in FIG. 2B.

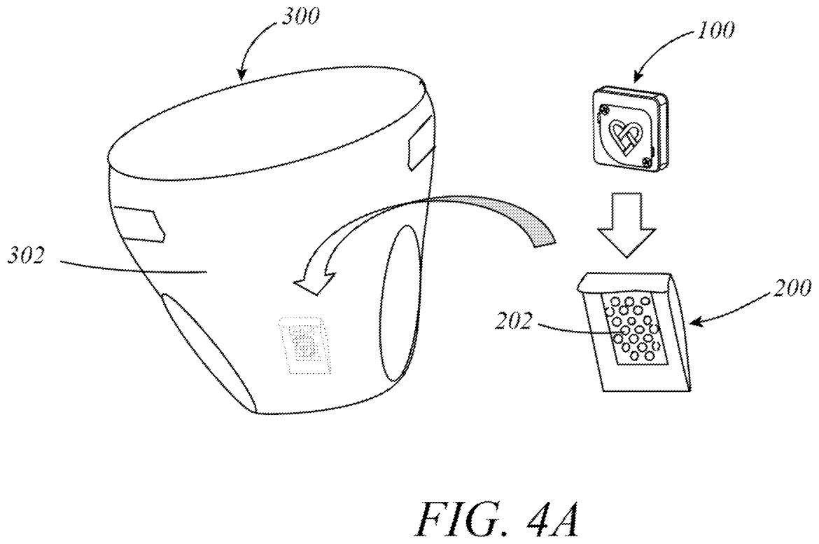

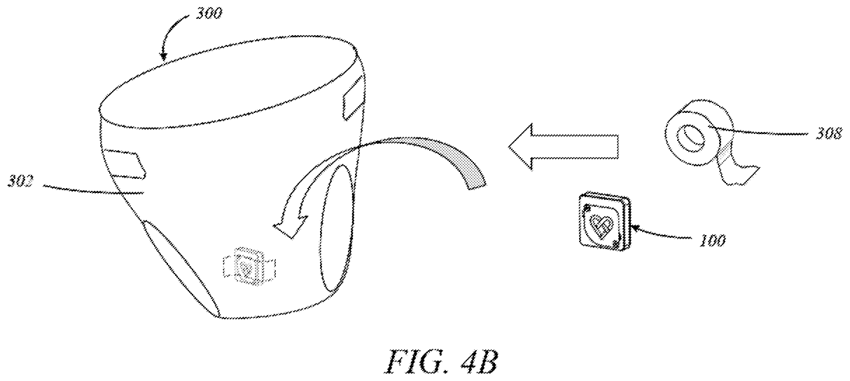

Referring generally to FIG. 4A, which illustrates a wireless module 100 being place into a secondary pouch 200 and then into an undergarment 300. An undergarment 300 can be defined as, but not limited to, an absorbent article, an incontinence undergarment, a diaper, an article of clothing, shorts, underwear, or the like. In some embodiments, the wireless module 100 can be removably attached using medical tape to an undergarment 300 or removably placed into a secondary pouch 200 wherein the secondary pouch can be removably attached to an undergarment 300 as shown in FIG. 4B. The secondary pouch 200 can comprise a plurality of thru holes 202 which can be thru either one or both of its surfaces to allow fluids or humidity to enter the inside of the secondary pouch and allow the wireless module to detect a saturation event. The secondary pouch 200 can have an adhesive backing that can be removably attached to the undergarment's surface 302. The secondary pouch 200 can be made out various materials including, but not limited to, plastics, linens, silk, cotton, or the like, and can be in various shapes such as, but not limited to, circle, square, rectangle, or the like. In another embodiment, a secondary pouch 200 can be omitted, and the wireless module 100 can have an adhesive backing and can be removably attached to the undergarment's surface 302.

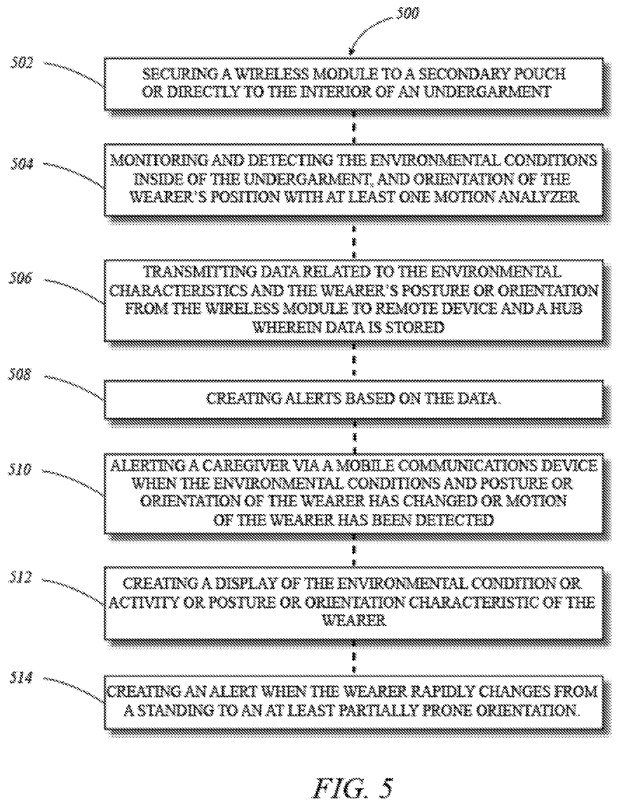

FIG. 5 illustrates a process flow diagram 500 depicting a method for detecting the state of the wireless module 100, in accordance with one, or more embodiments. It is noted herein that the process 500 may be implemented by various embodiments of the wireless module 100. It is further recognized, however, that the process 500 is not limited to the architecture of the wireless module 100. In step 502, a wireless module 100 can be secured to the interior of an undergarment, or in other embodiments secured into a secondary pouch 200 wherein the secondary pouch is attached to the undergarment 300. The secondary pouch 200 can be removable and can be replaced in the undergarment 300 when soiled. In step 504, monitoring and detecting the environmental conditions inside of the undergarment 302 with an environmental sensor 112, and detecting the posture or orientation of the wearer's orientation with at least one motion analyzer 116.

In step 506, transmitting data related to the environmental characteristics and the wearer's posture or orientation from the wireless module to remote device and a hub wherein the data is stored. In some embodiments, a controller 110 can determine the difference between insertion, and removal of the wireless module 100 from the undergarment 302 and then determine the presence or the lack of humidity. For example, the temperature and humidity sensor 112 can detect the humidity characteristics (e.g., via measurement of relative or absolute humidity) of the condition of the undergarment. In some embodiments, the controller 110 can detect the change in temperature when inserted into the undergarment 300, or removed from the undergarment wherein the temperature can be associated with the wearer's body temperature, fluid temperature, and/or ambient temperature when removed. A controller 110 can monitor and detect motion, posture or orientation of the wearer wherein the motion analyzer 116 can detect the motion, posture or orientation characteristic (e.g., x, y, z, positional information of the controller). For example, the controller 110 may detect an angle of orientation of the wearer with respect to the ground, or the surface they are located on, and detect whether there is an acceleration event such as a fall. In certain embodiments, the controller can detect a wearer's fall event by measuring the wearer's static location, and comparing it to an acceleration event detected by the motion analyzer 116.

In step 508, creating alerts based on the data. The wireless module can transmit environmental characteristics, and the wearer's motion, posture or orientation from the wireless module to a central hub, or cloud server/database wherein the data and events are stored, alerts are created and then transmitted to a wireless device, or web application. At step 510, alerting a caregiver via a mobile communications device when the environmental conditions and posture or orientation of the wearer has changed or motion of the wearer has been detected. In some embodiments, the last known environmental condition of the undergarment 300 and motion, posture or orientation of the wearer can be stored wherein the last known condition can be determined, transmitted, and then reset to a dry or static condition. At step 512, creating a display of the environmental conditions or activity or posture or orientation characteristic of the wearer. A caregiver can be alerted through a display on a module device, remote communications device, or a personal computer when the environmental conditions in the wearer's undergarment and/or motion, posture or orientation of the wearer has changed. For example, the wireless module 100 can transmit data collected to a wireless hub wherein the wireless hub relays a string of instructions to the secured cloud which can determine what event has occurred, and then that known event can be relayed to the caregiver. At step 514, creating an alert when the wearer rapidly changes from a standing position to an at least partially prone orientation. A wireless module's 100 can update its software remotely. For example, implementations may include an update or modification of the firmware, programmable hardware, such as by performing a reception of or a transmission of one or more instructions in relation to one or more operations described herein. Alternatively, or additionally, in some variants, an implementation may include special purpose components, or added components. Specifications or other implementations may be transmitted by one or more instances through the central hub, or pushed through the secured cloud, through the central hub, to the wireless module 100.

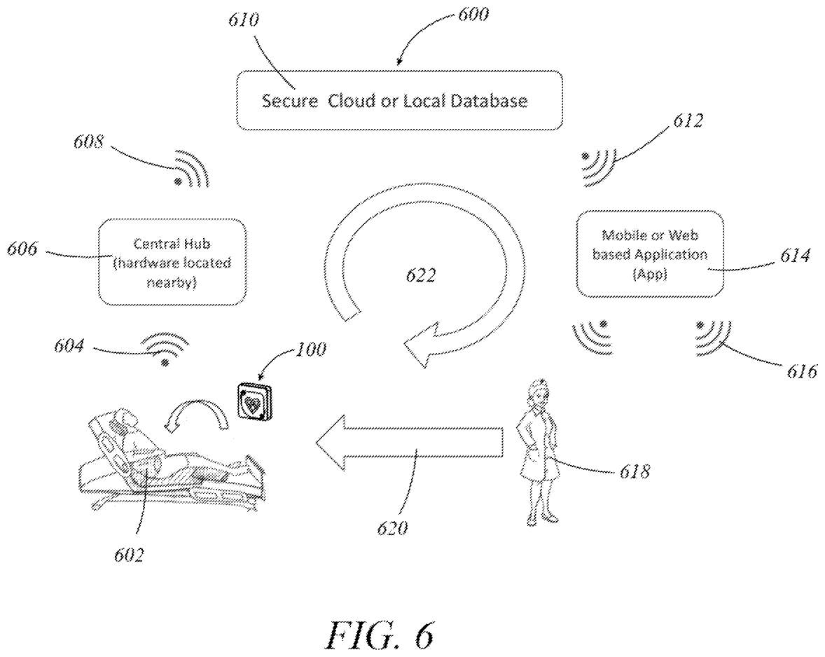

FIG. 6 illustrates a high level schematic view of a method and system for monitoring, detecting and alerting caregivers when humidity or saturation is detected in an undergarment, or the wearer's motion has been detected or when the wearer's posture or orientation has changed. A circle 622 represents a conceptual depiction of the process for monitoring, detecting, and alerting when humidity or saturation is detected in an undergarment using a wireless module 100. A wireless module 100 can be placed and secured into an individual's undergarment 602. The wireless module 100 using a controller 110 having a communications device can transmit a signal 604 to a central hub 706 that can be located within the individual's room, hallway, or caregiver's station. In certain embodiments, a central hub 606 can be omitted and the wireless module 100 can transmit a signal to a cell phone, a mobile communication device, a LAN, or secured cloud. The central hub 606 can send a signal 608 to a secured cloud, or local database system 610 wherein the secured cloud system 610 can store the data and events sent by the wireless module. Those having skill in the art will appreciate that there are various vehicles by which a process and/or system and/or technologies described herein can be effected by hardware, software, and/or firmware. The preferred vehicle can vary with the context in which the processes and/or systems and/or other technologies are deployed. For example, if the implementer determines that it is more efficient to bypass a central Hub, and go with mainly a software implementation with Wi-Fi capabilities, or opt for a mainly hardware and/or firmware vehicle the implementer has that option. Hence, there are several different ways by which the processes and/or device, and/or technologies described herein may be effected, none of which is inherently superior to the other. Other embodiments may include, for example, updates or modifications to software or firmware, or of gate arrays or programmable hardware, such as performing a task to monitor, detect, and then transmit an environmental condition of the undergarment, or motion, posture or orientation of the wearer. In some embodiments, described herein, logic and similar implementations may include software or other control structures. Electronic circuitry, for example, may have one or more paths of electrical current constructed and arranged to implement various functions as described herein.

In some embodiments, the secured cloud system 610 can create alerts which then communicate 612 to a mobile or web based application (“App”) 614 using a computing device wherein information is communicated wirelessly via for example, but not limited to, a BLUETOOTH Low Energy (BLE), BLUETOOTH module, Wi-Fi module, IEEE 802.15.4, Z-Wave, Single/Dual Mode Radio Chip, cellular networks, or the like on a local cloud server 610 to remote computing devices 614, wherein the alerts and graphical representations of the historical data, and events are displayed to a caregiver's 618 cellular phone, computer, tablet, or the like 616 wherein a caregiver can be for example, but not limited to, nurse, parent, caregiver, nanny, babysitter, or the like. Information transmitted to the caregiver can include the identity of the user of the wireless module 100, the location of the user, other information from the user's profile, the nature of the change event, or time elapsed since the change event.

In certain embodiments, the wireless module 100 is designed to recognize when an undergarment is wet or saturated. When the wireless module 100 can no longer communicate with the central hub 606 the caregiver 618 receives a notification that the wireless module is either out of range, the patient has moved, or the wireless module is out of power and the batteries need to be recharged or replaced. In a general sense, those that are skilled in the art will recognize that the various aspects described herein can be implemented individually, or as any combination thereof can be viewed as being composed as a “controller” which includes electrical circuitry. The control, or electrical circuitry includes, but not limited to having at least one discrete electrical circuit, electrical circuitry having at least one discrete electrical circuit, electrical circuitry having at least one application specific integrated circuit, electrical circuitry forming a general-purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, Wireless LAN, Central HUB, optical-electrical equipment, etc.). Those having skill in the art will recognize that the subject matter described herein may be implemented in an analog or digital or some combination thereof.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

What is claimed is:

1. A wireless module for use in an environment and motion, posture or orientation detecting system, wherein the environment has at least one environmental feature, the wireless module comprising:
a controller accommodated within a housing at least partially separating the controller from the environment;
an environmental sensor at least partially in contact with the environment to detect at least one of the environmental features; and
a motion sensor to detect motion, posture or orientation of a wearer of the wireless module.

2. The wireless module according to claim 1, wherein the controller further comprises a transceiver, and a memory medium wherein the controller is suitable for executing programmed instructions stored on the memory medium, wherein program instructions are configured to cause the transceiver to transmit information to a remote device about at least one environmental feature and the motion, posture or orientation of the wearer of the wireless module.

3. The wireless module according to claim 1, wherein at least one environmental feature is selected from the group comprising humidity, temperature, and the presence of a compound.

4. The wireless module according to claim 1, wherein the environmental sensor is within the housing and wherein the housing has at least one thru hole substantially positioned over the environmental sensors, wherein the wireless module further comprises a bushing substantially positioned between the environmental sensors and the housing.

5. The wireless module according to claim 4, wherein the wireless module further comprises a filter element, and a locking ring wherein the filter element is affixed into the bushing and the locking ring secures the filter element substantially between the housing, and the locking ring.

6. The wireless module according to claim 1, wherein the housing comprises a cover encapsulating the controller and a power supply wherein the wireless module is resistant to fluids.

7. The wireless module according to claim 1, wherein the wireless module is removably attached to an undergarment using medical tape or is secured into a secondary pouch wherein the secondary pouch can be removably attached to an undergarment, wherein the secondary pouch has a plurality of holes on its surface to allow fluids to penetrate the secondary pouch.

8. The wireless module according to claim 1, further comprising a battery, wherein the battery is rechargeable or replaceable and wherein the controller is configured to save power during operation by intermittently measuring various characteristics and processing data.

9. The wireless module according to claim 2, wherein the remote device is a cell phone or a mobile communications device or a wireless communication computer.

10. A system according to claim 1, wherein the motion sensor is at least one of an accelerometer or a compass.

11. A system according to claim 2, wherein the program instructions are further configured to compare the state of the undergarment to determine whether there is an environmental change with the environmental sensors, and then transmit that state to a central hub.

12. A method of detecting wetness or saturation and motion, posture or orientation of an undergarment comprising:
securing a wireless module to a secondary pouch or directly to an interior of an undergarment;
monitoring environmental conditions inside of the undergarment with an environmental sensor, and an orientation characteristic of a wearer's position with at least one motion analyzer;
transmitting data related to the environmental characteristics and the wearer's posture or orientation from the wireless module to remote device and a hub wherein the data is stored,
creating alerts based on the data; and;
alerting a caregiver via a mobile communications device when the environmental conditions and posture or orientation of the wearer has changed or motion of the wearer has been detected.

13. The method of claim 12, further comprising storing a last known environmental condition, or posture or orientation characteristic and creating an alert based on a change in the environmental condition or posture or orientation characteristic.

14. The method of claim 12, further comprising:
creating a display of the environmental condition or activity or posture or orientation characteristic of the wearer.

15. The method of claim 12, wherein an alert is created when the wearer rapidly changes from a standing to an at least partially prone orientation.

16. The method of claim 12, wherein the caregiver is alerted when the undergarment changes from dry to wet or saturated.

17. A method of claim 12, wherein the mobile communications device comprises at least one, smartphone, personal digital assistance, a tablet device, a table PC, a laptop, a smart book, or an ultra-book.

18. A method of claim 12, wherein the wireless module can be updated remotely through the hub.
